(19) United States
(12) Patent Application Publication
(10) Pub. No.: US 2008/0177182 A1
(43) Pub. Date: Jul. 24, 2008

(54) ULTRASONIC IMAGING APPARATUS AND METHOD FOR ACQUIRING ULTRASONIC IMAGE

(75) Inventors: Masao Takimoto, Otawara-shi (JP); Muneki Kataguchi, Nasushiobara-shi (JP); Fumiyasu Sakaguchi, Otawara-shi (JP); Akihiro Kakee, Nasushiobara-shi (JP); Atsushi Sumi, Otawara-shi (JP); Shigemitsu Nakaya, Otawara-shi (JP)

Correspondence Address: OBLON, SPIVAK, MCCLELLAND MAIER & NEUSTADT, P.C., 1940 DUKE STREET, ALEXANDRIA, VA, 22314, US

(73) Assignees: KABUSHIKI KAISHA TOSHIBA, Tokyo (JP); TOSHIBA MEDICAL SYSTEMS CORPORATION, Otawara-Shi (JP)

(21) Appl. No.: 11/944,546

(22) Filed: Nov. 23, 2007

(30) Foreign Application Priority Data
Jan. 24, 2007 (JP) 2007-013712

(51) Int. Cl. A61B 8/00

(52) U.S. Cl. 600/441

(57) ABSTRACT

A display controller causes a display to display a 3-dimensional B-mode image and a 3-dimensional colored Doppler image in a superimposed state. A marker generator generates three planar markers that are orthogonal to each other, and the display controller causes the display to display the three planar markers so as to be superimposed on the 3-dimensional B-mode image and so on. An image generator generates 3-dimensional B-mode image data of a region other than a region existing on a viewpoint side of regions sectioned by the three planar markers. Further, the image generator generates 3-dimensional colored Doppler image data for the region on the viewpoint side. For the region existing on the viewpoint side, only a 3-dimensional colored Doppler image is displayed on the display. An intersection of the three planar markers is set as a sample marker, and Doppler data of the intersection is acquired.

### BACKGROUND OF THE INVENTION

1. Field of the Invention

The present invention relates to an ultrasonic imaging apparatus configured to transmit ultrasonic waves to a subject body and acquire the movement state of fluid inside the subject body by the Doppler effect, and a method for acquiring an ultrasonic image.

2. Description of the Related Art

For measurement of the speed of blood flow, an ultrasonic imaging apparatus capable of Doppler scan is used. Doppler scan is a technique for obtaining information on blood flow in a subject body on the basis of the principle of the ultrasonic Doppler method. An ultrasonic diagnostic apparatus adopts a method of observing a change of blood-flow information with time by the pulsed-wave Doppler method (PW Doppler method) or the continuous-wave Doppler method (CW Doppler method). For measurement of the speed of blood flow, the pulsed-wave Doppler method (PW Doppler method) is implemented in general.

In the case of implementing the pulsed-wave Doppler method, it is necessary to set a sample marker indicating a position to obtain blood-flow information, on a 2-dimensional B-mode tomographic image or a colored Doppler image.

For example, B-mode scan is performed by using a 1-dimensional array probe in which ultrasonic transducers are placed in a row in a scanning direction, whereby B-mode tomographic image data is acquired, and a B-mode tomographic image of a 2-dimensional image is displayed on a display device. Further, a colored Doppler image may be displayed simultaneously when the B-mode tomographic image is displayed. At the time of Doppler scan, a movable sample marker is displayed on the B-mode tomographic image, and the operator designates a position to obtain blood-flow information with the sample marker. When the desired position is designated with the sample marker and Doppler scan is performed, Doppler information (blood-flow information) at the designated position is obtained. Doppler data representing the blood-flow information is shown with time taken on the horizontal axis and speed (frequency) taken on the vertical axis. In general, Doppler data is shown on a display device simultaneously when the B-mode tomographic image is displayed. Here, the sample marker has a predetermined width that can be changed by the operator. In the pulsed-wave Doppler method, blood-flow information within an observation point having the predetermined width is acquired.

On the other hand, by using a 2-dimensional array probe in which ultrasonic transducers are placed 2-dimensionally, it is possible to spatially scan the inside of a subject body and acquire 3-dimensional biological information. Hereinafter, 3-dimensional scan may be referred to as “volume scan.” By performing volume scan, it is possible to 3-dimensionally display a diagnosis site within a 3-dimensional space. Also in volume scan, for acquisition of blood-flow information, it is necessary to display a sample marker as well as a 3-dimensional image on a display device and designate a position desired to obtain blood-flow information by using the sample marker (e.g. Japanese unexamined patent application publication JP-A 2006-180998, and Japanese unexamined patent application publication JP-A 2000-135217).

In the conventional arts, the sample marker is set in the 3-dimensional space by forming and displaying 2-dimensional images that are individually parallel to three orthogonal cross-sections, on the display device. In other words, three 2-dimensional images that are orthogonal to each other are formed and displayed on the display device. Then, the operator views each of the three 2-dimensional images in turn, and sets the sample marker on each of the three 2-dimensional images. Thus, the sample makers are set in the 3-dimensional space.

In the case of using the 2-dimensional array probe, however, it is possible to acquire a 3-dimensional image. Consequently, in order to acquire the Doppler information (blood-flow information), it is necessary to designate a single point in the 3-dimensional space on the screen of the display device. In the case of displaying the three 2-dimensional images on the display device, it is necessary to set the sample markers on the respective 2-dimensional images. Therefore, the operator needs an extremely complicated operation, as compared with using the 1-dimensional array probe, whereby it becomes difficult to set the sample marker on the desired position. As a result, there arises a problem that it takes long time to diagnose and the efficiency of an examination decreases.

Accordingly, such an ultrasonic imaging apparatus has been desired that the operator does not need to view each of 2-dimensional images of three cross-sections to set sample markers of the respective 2-dimensional images and can easily set a sample marker by viewing only a 3-dimensional image.

### SUMMARY OF THE INVENTION

An object of the present invention is to provide an ultrasonic imaging apparatus that allows simple designation of a position to acquire blood-flow information on a 3-dimensional image, and a method for acquiring an ultrasonic image.

In a first aspect of the present invention, an ultrasonic imaging apparatus comprises: a scanner configured to ultrasonically scan an inside of a subject body; an image generator configured to generate 3-dimensional B-mode image data showing a shape of the inside of the subject body and 3-dimensional colored Doppler image data showing blood flow, based on data acquired through scan by the scanner; a marker generator configured to generate a first planar marker, a second planar marker and a third planar marker that are movable and cross each other; and a display controller configured to cause a display to display the first planar marker, the second planar marker and the third planar marker so as to be superimposed on a 3-dimensional B-mode image based on the 3-dimensional B-mode image data and a 3-dimensional colored Doppler image based on the 3-dimensional colored Doppler image data, wherein: the display controller causes the display to display the 3-dimensional colored Doppler image in an attention region on a preset viewpoint side of regions sectioned by the first planar marker, the second planar marker and the third planar marker; the scanner receives coordinate information of an intersection of the first planar marker, the second planar marker and the third planar marker from the marker generator, and executes Doppler scan on a site corresponding to the intersection; and the image generator generates Doppler data showing blood-flow information at the intersection, based on data acquired through the Doppler scan.

According to the first aspect, only the 3-dimensional colored Doppler image is displayed in the attention region existing on the viewpoint side of the regions sectioned by the three planar markers. Therefore, by including a site desired to acquire blood-flow information in the region, it is possible to display the state of blood flow at the site so as to be easy to observe. Thus, since the site desired to acquire blood-flow information can be easily observed, it is possible to easily designate a position to acquire blood-flow information. Furthermore, by taking the intersection of the three planar markers as the position to acquire Doppler information (the position to set the sample marker), it is possible to easily designate a position to acquire blood-flow information.

Further, in a second aspect of the present invention, a method for acquiring an ultrasonic image comprises: ultrasonically scanning an inside of a subject body; generating 3-dimensional B-mode image data showing a shape of the inside of the subject body and 3-dimensional colored Doppler image data showing blood flow, based on data acquired through the scanning; causing a display to display a first planar marker, a second planar marker and a third planar marker that are movable and cross each other so as to be superimposed on a 3-dimensional B-mode image based on the 3-dimensional B-mode image data and a 3-dimensional colored Doppler image based on the 3-dimensional colored Doppler image data; causing the display to display the 3-dimensional colored Doppler image in an attention region on a preset viewpoint side of regions sectioned by the first planar marker, the second planar marker and the third planar marker; executing Doppler scan on a site corresponding to an intersection of the first planar marker, the second planar marker and the third planar marker; and generating Doppler data showing blood-flow information at the intersection, based on data acquired through the Doppler scan.

### BRIEF DESCRIPTION OF THE DRAWINGS

FIG. 1 is a block diagram showing an ultrasonic imaging apparatus according to an embodiment of the present invention.

FIG. 2 is a view showing a display example of a 3-dimensional B-mode image.

FIG. 3 is a view showing a display example of a marker having a planar shape.

FIG. 4 is a view showing a display example of a 3-dimensional colored Doppler image.

FIG. 5 is a view showing an example of rotation of a 3-dimensional image.

FIG. 6 is a top view showing an example of a user interface.

FIG. 7A is a view for explaining an operation and process for obtaining an angle used for angle correction of blood-flow information.

FIG. 7B is a view for explaining an operation and process for obtaining an angle used for angle correction of blood-flow information.

FIG. 8 is a flowchart for explaining a series of operations of the ultrasonic imaging apparatus according to the embodiment of the present invention.

### DETAILED DESCRIPTION OF THE EMBODIMENTS

### (Configuration)

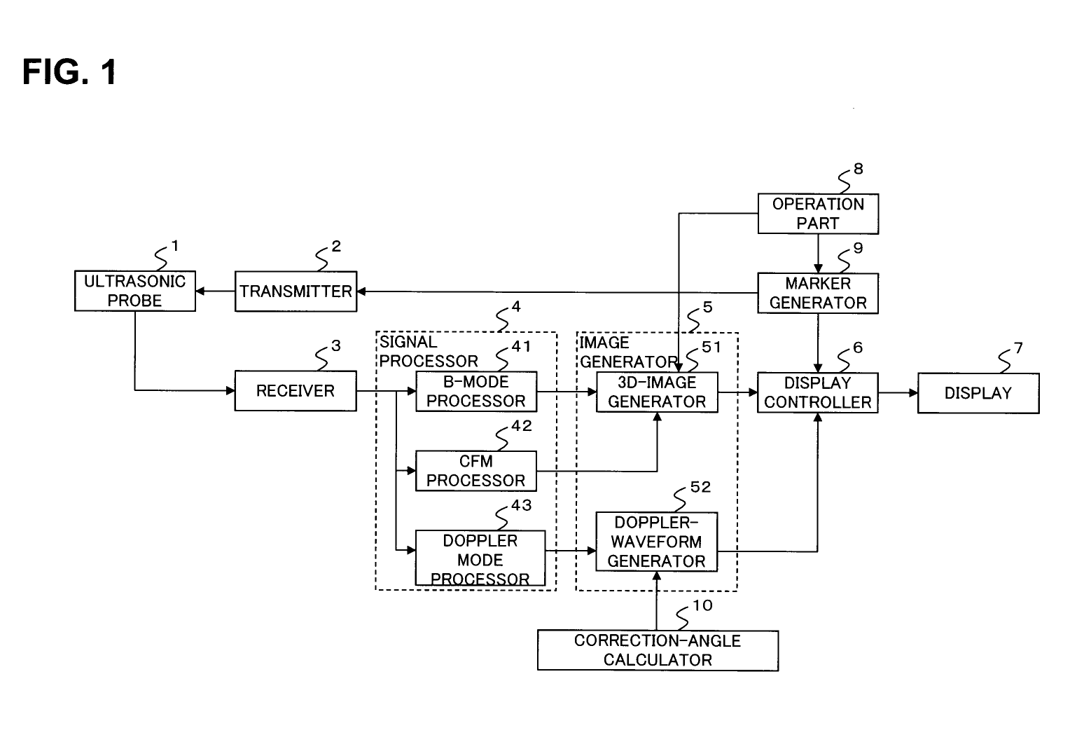

An ultrasonic imaging apparatus according to an embodiment of the present invention will be explained with reference to FIG. 1. FIG. 1 is a block diagram showing the ultrasonic imaging apparatus according to the embodiment of the present invention.

The ultrasonic imaging apparatus according to this embodiment is an apparatus that is operable in accordance with modes such as the B-mode to display a B-mode tomographic image, the M-mode to display a change with time of a position of a reflection source in an ultrasonic beam direction in the form of a motion curb, the Doppler mode (pulsed-wave Doppler (PW) or continuous-wave Doppler (CW)) to display blood-flow information, and the CFM (Color Flow Mapping) mode to display blood-flow information.

This embodiment describes capture of an image of the heart as one example of a diagnosis site. Specifically, this embodiment describes observation of backflow of blood at the valve of the heart.

A 2-dimensional array probe in which a plurality of ultrasonic transducers are placed 2-dimensionally is used for an ultrasonic probe 1. This ultrasonic probe 1 can acquire 3-dimensional biological information by performing volume scan. Meanwhile, a 1-dimensional array probe may be used for the ultrasonic probe 1 that can scan in a 3-dimensional space by swinging a plurality of ultrasonic transducers placed in one row in a scanning direction, into a direction (swinging direction) orthogonal to the scanning direction.

A transmitter 2 supplies electric signals to the ultrasonic probe 1 so as to generate ultrasonic waves. The transmitter 2 comprises a clock generation circuit, a transmission delay circuit and a pulsar circuit, which are not shown. The clock generation circuit generates clock signals to determine the transmission timing or transmission frequency of the ultrasonic signals. The transmission delay circuit performs transmission focus by delaying transmission of the ultrasonic waves. The pulsar circuit, which incorporates the same number of pulsars as individual channels corresponding to the respective ultrasonic transducers, generates drive pulses at delayed transmission timing, and supplies the pulses to the respective ultrasonic transducers of the ultrasonic probe 1.

A receiver 3 receives signals from the ultrasonic probe 1. The receiver 3 comprises a preamplifier circuit, an A/D conversion circuit and a received-signal delayer/adder circuit, which are not shown. The preamplifier circuit amplifies echo signals outputted from each ultrasonic transducer of the ultrasonic probe 1 for every receiving channel. The A/D conversion circuit performs A/D-conversion of the amplified echo signals. The received-signal delayer/adder circuit provides the echo signals after A/D-conversion with delay time required for determination of reception directionality, and adds the signals. By addition of the signals, reflection components from a direction corresponding to the reception directionality are emphasized.

One example of the “scanner” of the present invention comprises the ultrasonic probe 1, the transmitter 2 and the receiver 3.

A signal processor 4 comprises a B-mode processor 41, a CFM processor 42, and a Doppler mode processor 43. Data outputted from the receiver 3 is processed in one of the processors in a predetermined manner.

The B-mode processor 41 visualizes amplitude information of echoes and generates B-mode ultrasonic raster data from echo signals. Specifically, the B-mode processor 41 performs a band-pass Filtering process on signals transmitted from the receiver 3, and thereafter, detects the envelope curve of the output signals and compresses the detected data through logarithmic conversion.

The CFM processor 42 visualizes moving blood-flow information and generates color ultrasonic raster data. Blood-flow information contains information such as speed, distribution and power, and is acquired as binarized information. Specifically, the CFM processor 42 comprises a phase detection circuit, an MTI filter, an autocorrelator and a flow-speed/distribution computing unit. This CFM processor 42 performs a high-pass filtering process (MTI filter process) for separating blood-flow signals from tissue signals and obtains blood-flow information such as the speed, distribution and power of blood flow at many points through an autocorrelation process. In addition, the CFM processor 42 may execute nonlinear processing for reducing and cutting tissue signals.

The Doppler mode processor 43 generates blood-flow information by the pulsed-wave Doppler method (PW Doppler method) or the continuous-wave Doppler method (CW Doppler method). For example, because of use of pulsed waves, the pulsed-wave Doppler method enables detection of Doppler shift frequency components of a certain depth. Thus, the pulsed-wave Doppler method enables measurement of the speed at a specific site and the speed of blood flow. For signals transmitted from the receiver 3, the Doppler mode processor 43 extracts Doppler shift frequency components by detecting the phase of received signals in a sample marker (blood-flow viewpoint) having a predetermined size, and performs an FFT (Fast Fourier Transform) process, thereby generating a Doppler frequency distribution showing the blood-flow speed in the sample marker (blood-flow viewpoint).

An image generator 5 comprises a 3D-image generator 51 and a Doppler-waveform generator 52. The 3D-image generator 51 converts signal-processed data represented by a signal sequence obtained by scan, into data of a coordinate system based on space coordinates (scan conversion process). The 3D-image generator 51 performs scan conversion for signal-processed data outputted from the B-mode processor 41, thereby generating B-mode image data representing the tissue form of the scanned subject. The 3D-image generator 51 also performs a scan conversion process on signal-processed data outputted from the CFM processor 42, thereby generating colored Doppler image data (color flow mapping data).

For example, in a case where volume scan is executed and volume data is acquired, the 3D-image generator 51 subjects the volume data to volume rendering, thereby generating 3-dimensional B-mode image data (hereinafter referred to as “3-dimensional B-mode image data”) and 3-dimensional colored Doppler image data (hereinafter referred to as “3-dimensional colored Doppler image data”). Additionally, the 3D-image generator 51 may perform an MPR (Multi Planar Reconstruction) process on the volume data, thereby generating image data (MPR image data) of an arbitrary cross-section. Ultrasonic image data such as the 3-dimensional B-mode image data, the 3-dimensional colored Doppler image data and the MPR image data are outputted to a display controller 6.

The Doppler-waveform generator 52 generates Doppler data such as blood-flow speed information based on the signal-processed data outputted from the Doppler mode processor 43.

The display controller 6 receives ultrasonic image data such as the 3-dimensional B-mode image data, the 3-dimensional colored Doppler image data and the MPR image data from the 3D-image generator 51, and causes a display 7 to display a 3-dimensional B-mode image based on the 3-dimensional B-mode image data, a 3-dimensional colored Doppler image based on the 3-dimensional colored Doppler image data, or an MPR image based on the MPR image data. For example, when the display controller 6 receives the 3-dimensional B-mode image data and the 3-dimensional colored Doppler image data, the display controller 6 causes the display 7 to display the 3-dimensional colored Doppler image in the superimposed state on the 3-dimensional B-mode image. Additionally, when the display controller 6 receives Doppler data such as blood-flow information from the Doppler-waveform generator 52, the display controller 6 causes the display 7 to display the Doppler data simultaneously with the 3-dimensional B-mode image and the 3-dimensional colored Doppler image. Thus, Doppler data with time taken on the horizontal axis and speed (frequency) taken on the vertical axis is displayed on the display 7.

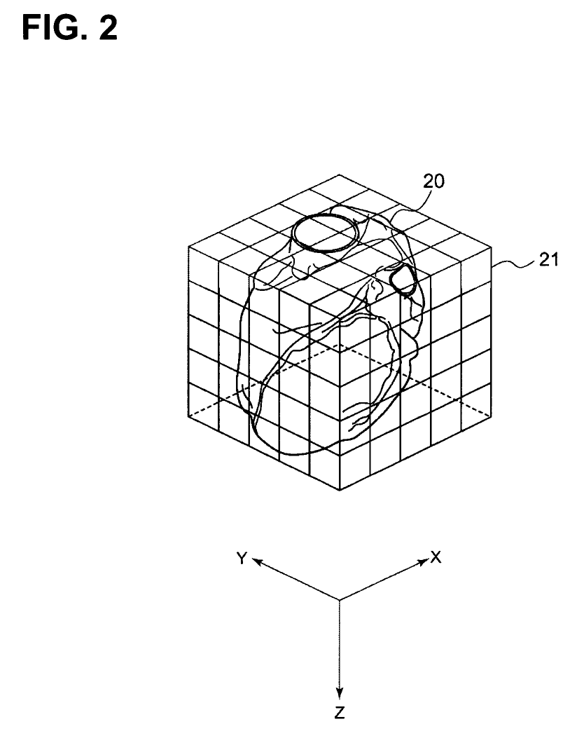

FIG. 2 shows an example of display of a 3-dimensional B-mode image. For example, as shown in FIG. 2, the display controller 6 causes the display 7 to display a 3-dimensional B-mode image 20, and further causes the display 7 to display a lattice-shaped auxiliary scale 21 so as to be superimposed on the 3-dimensional B-mode image 20. This auxiliary scale 21 is set so as to cover the whole 3-dimensional B-mode image 20.

A marker generator 9 generates a planar marker. In this embodiment, the marker generator 9 generates three planar markers that cross each other. For example, the marker generator 9 generates three planar markers that are orthogonal to each other. The display controller 6 causes the display 7 to display the planar markers so as to be superimposed onto the 3-dimensional B-mode image and the 3-dimensional colored Doppler image.

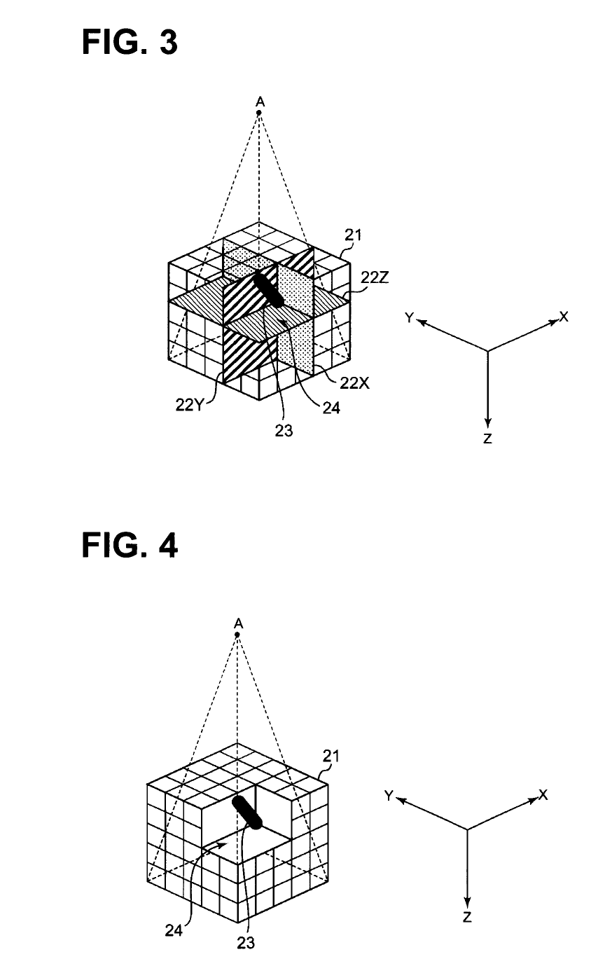

FIG. 3 shows an example of display of the planar markers. In this embodiment, the X axis (first axis), Y axis (second axis) and Z axis (third axis) make up an orthogonal coordinate system in which each axis is orthogonal to the others. The coordinate system of the planar markers coincides with the coordinate system of the 3-dimensional B-mode image and 3-dimensional colored Doppler image, and each of the coordinate systems is composed of the X axis, Y axis and Z axis shown in FIG. 3. For example, as shown in FIG. 3, the marker generator 9 generates a planar marker 22X orthogonal to the X axis, a planar marker 22Y orthogonal to the Y axis, and a planar marker 22Z orthogonal to the Z axis. The display controller 6 causes the display 7 to display the three planar markers 22X, 22Y and 22Z so as to be superimposed on the 3-dimensional B-mode image and 3-dimensional colored Doppler image.

The planar marker 22X can move along the X axis, the planar marker 22Y can move along the Y axis, and the planar marker 22Z can move along the Z axis. When the operator gives an instruction for movement of the planar markers 22X, 22Y and 22Z by using an operation part 8, the marker generator 9 generates new planar markers 22X, 22Y and 22Z whose display positions are changed, according to the movement instruction. The display controller 6 causes the display 7 to display the new planar markers 22X, 22Y and 22Z so as to be superimposed on the 3-dimensional B-mode image and 3-dimensional colored Doppler image. Additionally, the marker generator 9 may limit movement of the planar markers 22X, 22Y and 22Z by interval of a minimum scale on the auxiliary scale 21.

Further, as a default setting, the marker generator 9 may generate the planar markers 22X, 22Y and 22Z at positions dividing the 3-dimensional B-mode image into eight equal portions. For example, the marker generator 9 generates the planar markers 22X, 22Y and 22Z at positions dividing the whole lattice-shaped auxiliary scale 21 into eight equal portions. Thus, the display controller 6 causes the display 7 to display the planar markers 22X, 22Y and 22Z at the positions diving the auxiliary scale 21 into eight equal portions, as the default setting.

The 3D-image generator 51 receives coordinate information of the planar markers 22X, 22Y and 22Z from the marker generator 9, and generates 3-dimensional B-mode image data of a region other than a preset viewpoint-side region 24, of regions sectioned by the planar markers 22X, 22Y and 22Z. This region 24 corresponds to one example of the “attention region” of the present invention. To explain in more detail, the 3D-image generator 51 generates 3-dimensional B-mode image data of a region other than the region 24, which is nearest to the preset viewpoint, of the regions sectioned by the planar markers 22X, 22Y and 22Z.

The aforementioned viewpoint corresponds to a viewpoint designated by the operator in the volume rendering. Therefore, the 3D-image generator 51 generates 3-dimensional B-mode image data of a region other than the region 24 on the viewpoint side designated in the volume rendering, of the regions surrounded by the planar markers 22X, 22Y and 22Z. At this moment, the 3D-image generator 51 generates 3-dimensional colored Doppler image data in the region 24. Then, the display controller 6 causes the display 7 to display a 3-dimensional B-mode image based on the 3-dimensional B-mode image data of a region other than the region 24 and a 3-dimensional colored Doppler image based on the 3-dimensional colored Doppler image data of the region 24 so as to be superimposed onto each another.

FIG. 4 shows an example of display of a 3-dimensional colored Doppler image. When the 3D-image generator 51 generates the 3-dimensional colored Doppler image data of the region 24, as shown in, for example, FIG. 4, the display controller 6 causes the display 7 to display a 3-dimensional colored Doppler image 23 of the region 24. At this moment, the 3D-image generator 51 generates 3-dimensional B-mode image data of a region other than the region 24, and the display controller 6 causes the display 7 to display the 3-dimensional B-mode image of a region other than the region 24 and the 3-dimensional colored Doppler image of the region 24 so as to be superimposed onto each other. In other words, the display controller 6 causes the display 7 to simultaneously display the 3-dimensional B-mode image of a region other than the region 24 and the 3-dimensional colored Doppler image of the region 24.

For example, in the case of observing backflow of blood at the valve of the heart, the operator gives an instruction for movement of the planar markers 22X, 22Y and 22Z by using the operation part 8 while observing images displayed on the display 7 so that a site where the backflow is occurring is included into the region 24. In this way, the site where the backflow is occurring is displayed on the display 7 as a 3-dimensional colored Doppler image 23.

Additionally, the 3D-image generator 51 may generate 3-dimensional colored Doppler image data of all the regions including the region 24. In this case, the display controller 6 causes the display 7 to display 3-dimensional colored Doppler images of all the regions and 3-dimensional B-mode images of the regions other than the region 24 so as to be superimposed.

Further, the transmitter 2 receives coordinate information of the region 24 from the marker generator 24, and scans only the region 24 in color mode. Then, the CFM processor 42 and the 3D-image generator 51 generate 3-dimensional colored Doppler image data of the region 24. The display controller 6 causes the display 7 to display a 3-dimensional colored Doppler image of the region 24 and a 3-dimensional B-mode image of a region other than the region 24 in the superimposed state.

Additionally, the transmitter 2 may scan all the regions including the region 24 in color mode. In this case, the 3D-image generator 51 extracts 3-dimensional colored Doppler image data of the region 24 from data acquired through the scan. The display controller 6 causes the display 7 to display a 3-dimensional colored Doppler image based on the extracted 3-dimensional colored Doppler image data.

For example, in the case of observation of backflow of blood at the valve of the heart, the speed of the backflow and the time phase at which the backflow occurs are predetermined. Based on the speed and time phase, the 3D-image generator 51 extracts 3-dimensional colored Doppler image data indicating the backflow of blood from data acquired through scan. The display controller 6 causes the display 7 to display a 3-dimensional colored Doppler image of the region 24 and a 3-dimensional B-mode image of a region other than the region 24 so as to be superimposed upon each another.

Furthermore, the 3D-image generator 51 may generate B-mode tomographic image data along faces surrounding the region 24, without generating 3-dimensional B-mode image data, even in regions other than the region 24. The display controller 6 causes the display 7 to display a B-mode tomographic image based on the B-mode tomographic image data.

Additionally, the 3D-image generator 51 changes the view direction and performs rendering in accordance with a rotation instruction sent from the operation part 8, thereby generating 3-dimensional B-mode image data and 3-dimensional colored Doppler image data with the view direction changed. The display controller 6 causes the display 7 to display a 3-dimensional B-mode image and 3-dimensional colored Doppler image with the view direction changed.

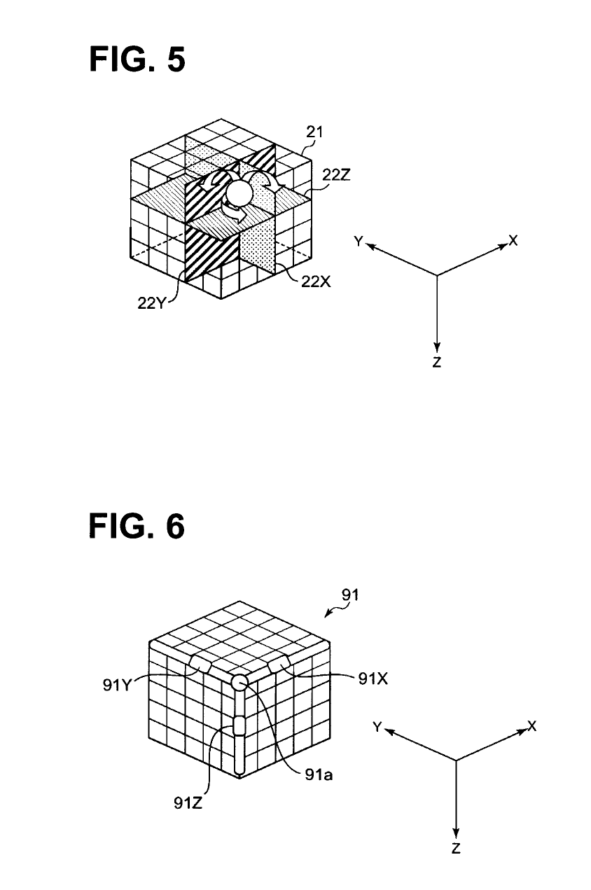

FIG. 5 shows one example of rotation of a 3-dimensional image. By taking the intersection of the planar markers 22X, 22Y and 22Z as the rotation center of the 3-dimensional image, as shown in, for example, FIG. 5, the operator can easily understand the rotation state.

The intersection of the planar markers 22X, 22Y and 22Z is set to the position of the sample marker by the controller (not shown). When the operator gives an instruction for execution of Doppler scan by using the operation part 8, the transmitter 2, in accordance with the instruction from the controller, receives coordinate information of the intersection of the planar markers 22X, 22Y and 22Z from the marker generator 9, and executes Doppler scan on a site corresponding to the coordinates of the intersection by the pulsed-wave Doppler method. Then, the Doppler mode processor 43 generates a Doppler frequency distribution representing the blood-flow information of the site corresponding to the intersection based on reception signals acquired through the Doppler scan. The Doppler-waveform generator 52 generates Doppler data representing a change of blood-flow speed with time, based on the Doppler frequency distribution. Upon reception of the Doppler data from the Doppler-waveform generator 52, the display controller 6 causes the display 7 to display the Doppler data. At this moment, the display controller 6 may cause the display 7 to display the Doppler data together with a 3-dimensional B-mode image and a 3-dimensional colored Doppler image.

For example, at the time of observation of the backflow of blood at the valve of the heart, while observing images displayed on the display 7, the operator moves the planar markers 22X, 22Y and 22Z by using the operation part 8 so that a site where the backflow is occurring is included in the region 24. By including the site where the backflow is occurring in the region 24, the site where the backflow is occurring is displayed on the display 7 as the 3-dimensional colored Doppler image 23. Consequently, the operator can observe the site where the backflow is occurring in detail.

Furthermore, in a case where the backflow of blood is occurring, in general, the sample marker is set to the part of the backflow, and Doppler data of the part of the backflow is acquired. For this, the operator observes the 3-dimensional colored Doppler image 23 showing the backflow of blood, and moves the intersection of the planar markers 22X, 22Y and 22Z to the position of the part of the backflow by using the operation part 8. Thus, the sample marker is set to the position of the part of the backflow, and the Doppler data of the part of the backflow is acquired.

From the above, according to this embodiment, in the region 24 existing on the viewpoint side of the regions surrounded by the planar markers 22X, 22Y and 22Z, the 3-dimensional B-mode image is not displayed, and only the 3-dimensional colored Doppler image 23 is displayed. Therefore, by moving the planar markers 22X, 22Y and 22Z so that a site desired to acquire blood-flow information is included in the region 24, it is possible to display the blood-flow state of the site so as to be easy to observe.

Besides, since the intersection of the planar markers 22X, 22Y and 22Z is set to the position of the sample marker, the operator needs merely to give an instruction for movement of the planar markers 22X, 22Y and 22Z and designate a position desired to acquire blood-flow information based on the intersection, with the result that the operator can easily designate the position desired to acquire the blood-flow information.

The operation part 8 is composed of a keyboard, a mouse, a trackball, a TCS (Touch Command Screen), or the like. The operator can set a projection direction of projecting light for volume data (a view direction) and Region Of Interest (ROI) by using the operation part 8.

Further, the operation part 8 comprises a user interface for moving the planar marker 22X along only the X axis, the planar marker 22Y along only the Y axis, and the planar marker 22Z along only the Z axis. The structural outline of this user interface is shown in FIG. 6. FIG. 6 is a top view showing one example of the user interface.

A user interface 91 comprises finger grips 91X, 91Y and 91Z. The finger grips 91X, 91Y and 91Z are placed at intervals of 120 degrees, and are movable linearly in a radial direction about a center portion 91a. The finger grip 91X is an interface for moving the planar marker 22X along the X axis. The finger grip 91Y is an interface for moving the planar marker 22Y along the Y axis. The 91Z is an interface for moving the planar marker 22Z along the Z axis.

For example, when the operator moves the finger grip 91X linearly, the marker generator 9, in accordance with a movement distance of the finger grip 91X, generates a new planar marker 22X at a display position corresponding to the movement distance. The display controller 6 causes the display 7 to display the new planar marker 22X. In this way, the markers 22X, 22Y and 22Z can be moved by the finger grips 91X, 91Y and 91Z that are movable linearly. The finger grips 91X, 91Y and 91Z can move only linearly, and have a smaller degree of freedom of movement than a trackball or the like, so that it becomes possible to move the planar markers 22X, 22Y and 22Z to desired positions with ease.

Furthermore, in the case of a Doppler examination, it is necessary to correct blood-flow information based on an angle formed by the ultrasonic-transmission direction and the blood-flow direction. In the case of displaying a 2-dimensional tomographic image and setting a sample marker on the tomographic image to acquire blood-flow information, the operator operates an angle marker for angle correction and makes the blood-flow direction and the angle marker parallel, thereby obtaining the angle formed by the blood-flow direction and the ultrasonic-transmission direction and correcting the blood-flow information based on the angle. Thus, in the case of setting an angle marker for a 2-dimensional tomographic image, it is easy to set the angle marker because a tomographic image is planar. However, it has been difficult to set the angle marker for a 3-dimensional colored Doppler image because the image has depth.

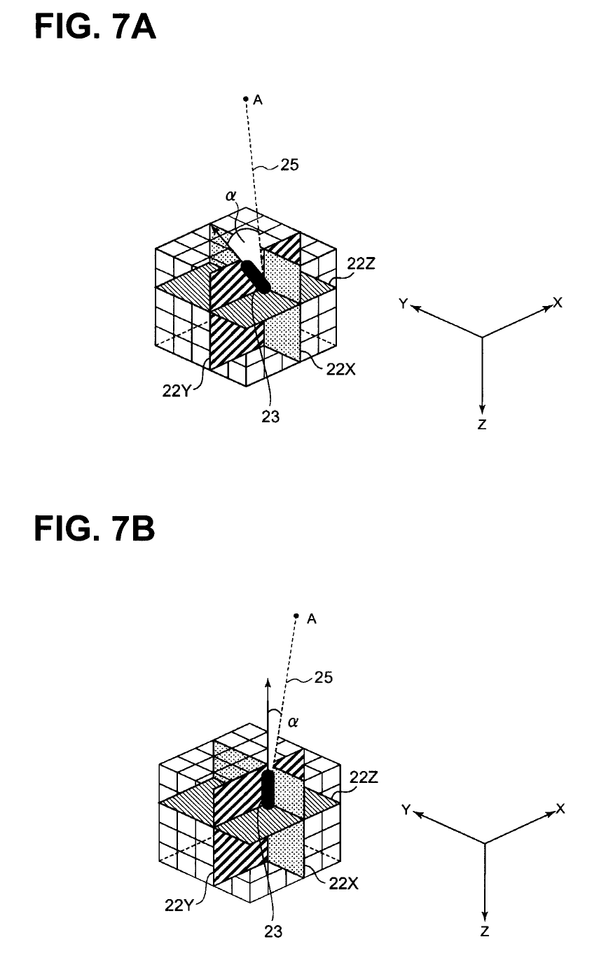

Accordingly, in this embodiment, a correction-angle calculator 10 is provided so that the angle formed by the direction of blood flow shown in the 3-dimensional colored Doppler image and the ultrasonic-transmission direction can be obtained. The correction-angle calculator 10 obtains an angle α between the direction of blood flow shown in the 3-dimensional colored Doppler image and the ultrasonic-transmission direction. This angle α is used for angle correction of blood-flow information. The operation and process to obtain the angle α will be explained with reference to FIG. 7A and FIG. 7B. FIG. 7A and FIG. 7B are views for explaining the operation and process of obtaining the angle used for angle correction of blood-flow information.

In FIG. 7A and FIG. 7B, a point A represents a source of ultrasonic waves (a ultrasonic transducer). For example, in a case where the blood flow shown in the 3-dimensional colored Doppler image 23 is displayed diagonally to the Z axis as shown in FIG. 7A, the operator gives an instruction for rotation by using the operation part 8 so that the direction of the blood flow shown in the 3-dimensional colored Doppler image 23 coincides with the Z axis as shown in FIG. 7B. Because the X axis, Y axis and Z axis are displayed on the display 7, the operator can adjust the direction of the blood flow shown in the 3-dimensional colored Doppler image 23 to the Z axis, while observing the Z axis and the 3-dimensional colored Doppler image 23 displayed on the display 7. This enables the operator to easily adjust the direction of the blood flow shown in the 3-dimensional colored Doppler image 23 so as to coincide with the Z axis.

Upon reception of the rotation instruction from the operation part 8, the 3D-image generator 51 changes the view direction and executes rendering in response to the rotation instruction, thereby generating new 3-dimensional colored Doppler image data in which the direction of the blood flow coincides with the Z axis. The display controller 6 causes the display 7 to display a 3-dimensional colored Doppler image based on the new 3-dimensional colored Doppler image data. Consequently, as shown in FIG. 7B, the direction of the blood flow shown in the 3-dimensional colored Doppler image 23 coincides with the direction of the Z axis.

Further, an ultrasonic-transmission direction 25 for the 3-dimensional colored Doppler image 23 is fixed, and the rotation operation changes the direction of the ultrasonic-transmission direction 25 with respect to the Z axis by the amount of the rotation. Upon reception of information on a rotation angle given in the operator's rotation instruction from the operation part 8, the correction-angle calculator 10 obtains the angle between the ultrasonic-transmission direction 25 and the Z axis after the rotation, based on the angle between the ultrasonic-transmission direction 25 and the Z axis before the rotation and based on the rotation angle. Since the Z axis coincides with the direction of the blood flow shown in the 3-dimensional colored Doppler image 23, the angle between the Z axis and the ultrasonic-transmission direction 25 after the rotation becomes equal to the angle α between the direction of the blood flow shown in the 3-dimensional colored Doppler image 23 and the ultrasonic-transmission direction 25. With this operation and process, the angle α between the direction of the blood-flow shown in the 3-dimensional colored Doppler image 23 and the ultrasonic-transmission direction 25 is obtained. In other words, since the angle between the Z axis and the ultrasonic-transmission direction 25 after the rotation becomes equal to the angle α, the Doppler-waveform generator 52 needs merely to correct the blood-flow speed based on the angle between the Z axis and the ultrasonic-transmission direction 25 after the rotation.

The Doppler-waveform generator 52 corrects the blood-flow speed by using the angle α between the 3-dimensional colored Doppler image 23 and the ultrasonic-transmission direction 25, and generates Doppler data with the corrected angle.

As described above, according to this embodiment, it becomes possible to easily obtain the angle α formed by the direction of the blood flow shown in the 3-dimensional colored Doppler image 23 and the ultrasonic-transmission direction 25, by making the direction of the blood flow shown in the 3-dimensional colored Doppler image 23 coincide with the Z axis, without a conventional operation of the angle marker. Consequently, it becomes possible to easily correct the blood-flow speed.

Further, by making the direction of the blood flow shown in the 3-dimensional colored Doppler image 23 coincide with the Z axis in a state where the Z axis is displayed on the display 7, visual understanding is facilitated, and the operator can easily set. Additionally, in this embodiment, by making the direction of the blood flow shown in the 3-dimensional colored Doppler image coincide with the Z axis by taking the Z axis as a reference axis, the angle α formed by the direction of the blood flow shown in the 3-dimensional colored Doppler image and the ultrasonic-transmission direction is obtained. The angle α may be obtained by taking the X axis or the Y axis as the reference axis instead of the Z axis.

The larger the angle α between the blood-flow direction and the ultrasonic-transmission direction becomes, the bigger an error in value of the blood-flow speed becomes. Therefore, a warning (e.g. an alarm) may be issued when the angle α becomes equal to or larger than a preset angle. For example, when the correction-angle calculator 10 compares the angle α with the preset angle and the angle α becomes equal to or larger than the preset angle, the correction-angle calculator 10 outputs an instruction for display of a warning to the display controller 6. The display controller 6 causes the display 7 to display the warning in accordance with the instruction. Meanwhile, a speaker or the like may be installed, which generates a warning sound in accordance with the instruction for warning. The preset angle is, for example, 60° or 70°. Therefore, a degree of 60° or 70° is previously set as the preset angle in the correction-angle calculator 10, and when the angle α becomes equal to or larger than 60° or 70°, the warning is displayed, or the warning sound is generated. This warning allows the operator to recognize that an error in blood-flow information having been acquired already is large.

Further, the ultrasonic diagnostic apparatus comprises a controller (not shown). The controller is connected to the respective parts of the ultrasonic diagnostic apparatus, and controls the respective parts. In this embodiment, the controller sets the intersection of the planar markers 22X, 22Y and 22Z as a position to acquire Doppler information (a sample marker). When the operator gives an instruction for execution of Doppler scan by the operation part 8, the controller causes the transmitter 2 to execute Doppler scan on the position in accordance with the execution instruction.

Additionally, each process executed by the 3D-image generator 51, the Doppler-waveform generator 52, the display controller 6, the marker generator 9 and the correction-angle calculator 10 may be executed through hardware or software.

For example, the 3D-image generator 51, the Doppler-waveform generator 52, the display controller 6, the marker generator 9 and the correction-angle calculator 10 are composed of a CPU and a memory device such as ROM, RAM and HDD. The memory device stores a 3D-image generating program for a function of the 3D-image generator 51, a Doppler-waveform generating program for a function of the Doppler-waveform generator 52, a marker generating program for a function of the marker generator 9, a display control program for a function of the display controller 6, and a correction-angle calculating program for a function of the correction-angle calculator 10.

Then, when the CPU executes the 3D-image generating program stored in the memory device, the function of the 3D-image generator 51 is implemented; when the CPU executes the Doppler-waveform generating program, the function of the Doppler-waveform generator 52 is implemented; when the CPU executes the marker generating program, the function of the marker generator 9 is implemented; when the CPU executes the display control program, the function of the display controller 6 is implemented; and when the CPU executes the correction-angle calculating program, the function of the correction-angle calculator 10 is implemented.

### (Operations)

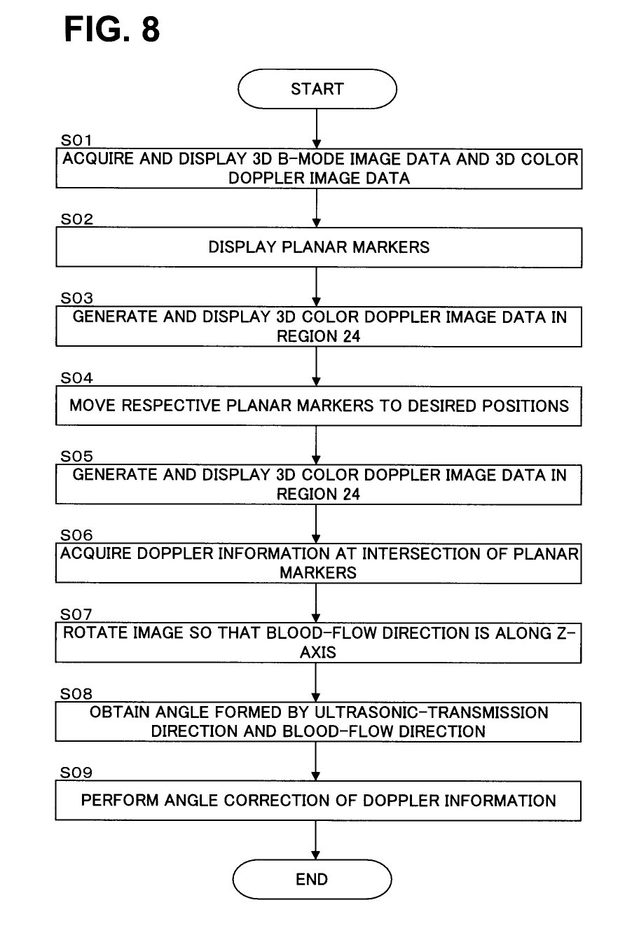

Next, the operations of the ultrasonic imaging apparatus according to the embodiment of the present invention will be explained with reference to FIG. 8. FIG. 8 is a flowchart for explaining a series of operations by the ultrasonic imaging apparatus according to the embodiment of the present invention. This embodiment describes observation of the backflow of blood at the valve of the heart.

### (Step S01)

First, a tomographic image of the inside of a subject body is acquired by using the ultrasonic probe 1, and the position of a diagnosis site is checked. After that, the operator gives an instruction for acquisition of a 3-dimensional B-mode image by using the operation part 8. When the instruction is given, the heart of the diagnosis site is scanned with ultrasonic waves by the ultrasonic probe 1, the transmitter 2 and the receiver 3. Then, 3-dimensional B-mode image data is generated by the B-mode processor 41 and the 3D-image generator 51. The display controller 6 causes the display 7 to display a 3-dimensional B-mode image based on the 3-dimensional B-mode image data. Furthermore, when the operator gives an instruction for acquisition of a 3-dimensional colored Doppler image by using the operation part 8, the heart is scanned with ultrasonic waves by the ultrasonic probe 1, the transmitter 2 and the receiver 3, and 3-dimensional colored Doppler image data is generated by the CFM processor 42 and the 3D-image generator 51. Then, the display controller 6 causes the display 7 to display the 3-dimensional B-mode image and the 3-dimensional colored Doppler image based on the 3-dimensional colored Doppler image data in the superimposed state.

### (Step S02)

Then, when the operator gives an instruction for start of Doppler scan by using the operation part 8, the marker generator 9 generates the planar markers 22X, 22Y and 22Z and, as shown in FIG. 3, the display controller 6 causes the display 7 to display the planar markers 22X, 22Y and 22Z superimposed on the 3-dimensional B-mode image and 3-dimensional colored Doppler image. At this moment, the planar markers 22X, 22Y and 22Z are displayed in the default positions.

### (Step S03)

Upon reception of coordinate information of the planar markers 22X, 22Y and 22Z from the marker generator 9, the 3D-image generator 51 generates 3-dimensional B-mode image data of the region other than the region 24 that is nearest to a preset viewpoint, of the regions sectioned by the planar markers 22X, 22Y and 22Z. Moreover, the 3D-image generator 51 generates 3-dimensional colored Doppler image data in the region 24. The display controller 6 causes the display 7 to display the 3-dimensional B-mode image based on the 3-dimensional B-mode image data of the region other than the region 24 and the 3-dimensional colored Doppler image based on the 3-dimensional colored Doppler image data of the region 24 in the superimposed state. For example, as shown in FIG. 4, the display controller 6 causes the display 7 to display the 3-dimensional colored Doppler image 23 of the region.

### (Step S04)

While observing the 3-dimensional colored Doppler image 23 displayed on the display 7 and the planar markers 22X, 22Y and 22Z, the operator gives an instruction for movement of the planar markers 22X, 22Y and 22Z by using the operation part 8 so that a site desired to observe is included in the region 24. The marker generator 9 generates new planar markers 22X, 22Y and 22Z moved in accordance with the movement instruction. The display controller 6 causes the display 7 to display the new planar markers 22X, 22Y and 22Z. For example, in the case of observation of the backflow of blood at the valve of the heart, the operator gives an instruction for movement of the planar markers 22X, 22Y and 22Z by using the operation part 8 so that a site where the backflow is occurring is included in the region 24. Consequently, the site where the backflow is occurring is displayed on the display 7 as the 3-dimensional colored Doppler image 23. For example, the operator gives an instruction for movement of the planar markers 22X, 22Y and 22Z by using a user interface 91 as shown in FIG. 6.

### (Step S05)

Then, the 3D-image generator 51 receives the coordinate information of the new planar markers 22X, 22Y and 22Z from the marker generator 9, and generates 3-dimensional B-mode image data included in the region other than the new region 24 and 3-dimensional colored Doppler image data included in the region 24. The display controller 6 causes the display 7 to display the 3-dimensional B-mode image based on the new 3-dimensional B-mode image data and the 3-dimensional colored Doppler image based on the new 3-dimensional colored Doppler image data in the superimposed state.

For example, in a case where the backflow of blood is occurring, a sample marker is set at the position of the part of the backflow in general, thereby acquiring Doppler data of the part of the backflow. In this embodiment, the intersection of the planar markers 22X, 22Y and 22Z is set as the sample marker, and therefore, the operator gives an instruction of movement of the planar markers 22X, 22Y and 22Z so that the intersection coincides with the part of the backflow. Thus, by moving the planar markers 22X, 22Y and 22Z so that the site where the backflow is occurring is included in the region 24, and further moving the intersection of the planar markers 22X, 22Y and 22Z to the vicinity of the port of the valve, an image taken from the proximity of the port of the valve can be shown in the 3-dimensional colored Doppler image 23 in the region 24. When recognition of the site where backflow of blood is occurring in the 3-dimensional colored Doppler image 23 is facilitated, the setting of the position of a 3-dimensional image is completed.

As described above, it is possible to set a sample marker at the intersection of the planar markers 22X, 22Y and 22Z while observing a complete picture in the 3-dimensional B-mode image and observing a site where backflow is occurring in the 3-dimensional colored Doppler image 23 of the region 24, so that it becomes possible to improve the efficiency of examinations.

### (Step S06)

When the position of the sample marker is set to the intersection of the planar markers 22X, 22Y and 22Z as described above, the coordinate information of the intersection is outputted from the marker generator 9 to the transmitter 2. The transmitter 2 executes Doppler scan on a site corresponding to the coordinates of the intersection, and Doppler data showing the blood-flow speed is generated by the Doppler mode processor 43 and the Doppler-waveform generator 52. The display controller 6 then causes the display 7 to display the Doppler data.

### (Step S07)

Further, for angle correction of blood-flow information, the operator gives an instruction for rotation of the 3-dimensional colored Doppler image 23 by using the operation part 8. Upon reception of the rotation instruction, the 3D-image generator 51 generates new 3-dimensional colored Doppler image data with the view direction changed, and the display controller 6 causes the display 7 to display a 3-dimensional colored Doppler image based on the new 3-dimensional colored Doppler image data. For example, as shown in FIG. 7A, if the blood flow shown in the 3-dimensional colored Doppler image 23 is displayed diagonally to the Z axis, the operator gives an instruction for rotation by using the operation part 8 so that the blood-flow direction shown in the 3-dimensional colored Doppler image 23 coincides with the Z axis as shown in FIG. 7B.

### (Step S08)

Upon reception of information on a rotation angle given in the operator's rotation instruction, the correction-angle calculator 10 obtains an angle between the ultrasonic-transmission direction 25 and the Z axis after the rotation, based on an angle between the ultrasonic-transmission direction 25 and the Z axis before the rotation and based on the rotation angle. Since the Z axis coincides with the blood-flow direction shown in the 3-dimensional colored Doppler image 23, the angle between the Z axis and the ultrasonic-transmission direction 25 coincides with the angle α between the direction of the blood-flow shown in the 3-dimensional colored Doppler image 23 and the ultrasonic-transmission direction 25.

### (Step S09)

Upon reception of information on the angle α between the blood flow shown in the 3-dimensional colored Doppler image 23 and the ultrasonic-transmission direction 25 from the correction-angle calculator 10, the Doppler-waveform generator 52 corrects blood-flow information (speed value of blood flow) by using the angle α, and generates angle-corrected Doppler data. The display controller 6 causes the display 7 to display the angle-corrected Doppler data.

Further, in a case where the angle α becomes equal to or larger than the predetermined angle (60° or 70°), the correction-angle calculator 10 outputs a warning instruction to the display controller 6, and the display controller 6 may cause the display 7 to display the fact that the angle of blood-flow direction is equal to or larger than the predetermined angle. Additionally, in a case where the angle α becomes equal to or larger than the predetermined angle, a warning sound may be generated by a speaker or the like.

The above process makes it possible to acquire Doppler information at the port of the valve and observe a blood-flow speed and the like. When measurement is performed based on the Doppler information, a series of examinations are completed.

What is claimed is:

1. An ultrasonic imaging apparatus comprising:
a scanner configured to ultrasonically scan an inside of a subject body;
an image generator configured to generate 3-dimensional B-mode image data showing a shape of the inside of the subject body and 3-dimensional colored Doppler image data showing blood flow, based on data acquired through scan by the scanner;
a marker generator configured to generate a first planar marker, a second planar marker and a third planar marker that are movable and cross each other; and
a display controller configured to cause a display to display the first planar marker, the second planar marker and the third planar marker so as to be superimposed on a 3-dimensional B-mode image based on the 3-dimensional B-mode image data and a 3-dimensional colored Doppler image based on the 3-dimensional colored Doppler image data, wherein:
the display controller causes the display to display the 3-dimensional colored Doppler image in an attention region on a preset viewpoint side of regions sectioned by the first planar marker, the second planar marker and the third planar marker;
the scanner receives coordinate information of an intersection of the first planar marker, the second planar marker and the third planar marker from the marker generator, and executes Doppler scan on a site corresponding to the intersection; and
the image generator generates Doppler data showing blood-flow information at the intersection, based on data acquired through the Doppler scan.

2. The ultrasonic imaging apparatus according to claim 1, wherein:
the image generator generates only the 3-dimensional colored Doppler image data in the attention region, the attention region being a region nearest to the preset viewpoint of the regions sectioned by the first planar marker, the second planar marker and the third planar marker; and
the display controller causes the display to display the 3-dimensional colored Doppler image in the attention region.

3. The ultrasonic imaging apparatus according to claim 1, wherein:
the marker generator generates the first planar marker, the second planar marker and the third planar marker that coincide with three axes of the 3-dimensional B-mode image and the 3-dimensional colored Doppler image and that are orthogonal to each other.

4. The ultrasonic imaging apparatus according to claim 1, wherein:
in accordance with an instruction for movement of the markers by the operator, the marker generator generates a new first planar marker moved along a first axis orthogonal to the first planar marker, generates a new second planar marker moved along a second axis orthogonal to the second planar marker, and generates a new third planar marker moved along a third axis orthogonal to the third planar marker;
the image generator generates only the 3-dimensional colored Doppler image data in the attention region of regions sectioned by the new first planar marker, the new second planar marker and the new third planar marker; and
the display controller causes the display to display the 3-dimensional colored Doppler image in the attention region.

5. The ultrasonic imaging apparatus according to claim 4, further comprising:
a user interface configured to give an instruction for movement of the first planar marker along only the first axis, an instruction for moving the second planar marker along only the second axis, and an instruction for movement of the third planar marker along only the third axis only.

6. The ultrasonic imaging apparatus according to claim 1, wherein:
in accordance with an instruction for rotation of an image by the operator, the image generator generates new 3-dimensional B-mode image data and new 3-dimensional colored Doppler image data with the viewpoint changed, by taking the intersection of the first planar marker, the second planar marker and the third planar marker as a rotation center; and
the display controller causes the display to display a 3-dimensional B-mode image based on the new 3-dimensional B-mode image data and a 3-dimensional colored Doppler image based on the new 3-dimensional colored Doppler image data in a superimposed state.

7. The ultrasonic imaging apparatus according to claim 1, wherein:
in accordance with an instruction for rotation of an image by the operator, the image generator generates new 3-dimensional colored Doppler image data in which a blood-flow direction shown by the 3-dimensional colored Doppler image data coincides with one of a direction of a first axis orthogonal to the first planar marker, a second axis orthogonal to the second planar marker and a third axis orthogonal to the third planar marker, and takes an angle formed by the direction of the coincident axis and an ultrasonic-transmission direction after the rotation as an angle α formed by a 3-dimensional colored Doppler image based on the new 3-dimensional colored Doppler image data and the ultrasonic-transmission direction, thereby obtaining Doppler data in which an angle is corrected by a degree of the angle α.

8. The ultrasonic imaging apparatus according to claim 7, further comprising:
an alarm configured to alert an operator that the angle α is equal to or larger than a preset angle, in a case where the angle α is equal to or larger than the preset angle.

9. A method for acquiring an ultrasonic image, comprising:
ultrasonically scanning an inside of a subject body;
generating 3-dimensional B-mode image data showing a shape of the inside of the subject body and 3-dimensional colored Doppler image data showing blood flow, based on data acquired through the scanning;
causing a display to display a first planar marker, a second planar marker and a third planar marker that are movable and cross each other so as to be superimposed on a 3-dimensional B-mode image based on the 3-dimensional B-mode image data and a 3-dimensional colored Doppler image based on the 3-dimensional colored Doppler image data;
causing the display to display the 3-dimensional colored Doppler image in an attention region on a preset viewpoint side of regions sectioned by the first planar marker, the second planar marker and the third planar marker;
executing Doppler scan on a site corresponding to an intersection of the first planar marker, the second planar marker and the third planar marker; and
generating Doppler data showing blood-flow information at the intersection, based on data acquired through the Doppler scan.

10. The method for acquiring an ultrasonic image according to claim 9, comprising:
generating only the 3-dimensional colored Doppler image data in the attention region, the attention region being a region nearest to the preset viewpoint of the regions sectioned by the first planar marker, the second planar marker and the third planar marker; and
causing the display to display the 3-dimensional colored Doppler image in the attention region.

11. The method for acquiring an ultrasonic image according to claim 9, wherein:
the first planar marker, the second planar marker and the third planar marker coincide with three axes of the 3-dimensional B-mode image and the 3-dimensional colored Doppler image, respectively.

12. The method for acquiring an ultrasonic image according to claim 9, comprising:
generating a new first planar marker moved along a first axis orthogonal to the first planar marker, generating a new second planar marker moved along a second axis orthogonal to the second planar marker, and generating a new third planar marker moved along a third axis orthogonal to the third planar marker, in accordance with an instruction for movement of the markers by the operator;
generating only the 3-dimensional colored Doppler image data in the attention region of regions sectioned by the new first planar marker, the new second planar marker and the new third planar marker; and
causing the display to display the 3-dimensional colored Doppler image in the attention region.

13. The method for acquiring an ultrasonic image according to claim 12, comprising:
causing a user interface to give the instruction for movement of the markers, the user interface giving an instruction for movement of the first planar marker along only the first axis, an instruction for movement of the second planar marker along only the second axis and an instruction for movement of the third planar marker along only the third axis.

14. The method for acquiring an ultrasonic image according to claim 9, comprising:
generating new 3-dimensional B-mode image data and new 3-dimensional colored Doppler image data with the viewpoint changed, by taking an intersection of the first planar marker, the second planar marker and the third planar marker as a rotation center, in accordance with an instruction for rotation of an image by the operator; and
causing the display to display a 3-dimensional B-mode image based on the new 3-dimensional B-mode image data and a 3-dimensional colored Doppler image based on the new 3-dimensional colored Doppler image data in the superimposed state.

15. The method for acquiring an ultrasonic image according to claim 9, comprising:
generating new 3-dimensional colored Doppler image data in which a blood-flow direction shown by the 3-dimensional colored Doppler image data coincides with one of a direction of a first axis orthogonal to the first planar marker, a second axis orthogonal to the second planar marker and a third axis orthogonal to the third planar marker in accordance with an instruction for rotation of an image by the operator, and taking an angle formed by the direction of the coincident axis and an ultrasonic-transmission direction after the rotation as an angle α between a 3-dimensional colored Doppler image based on the new 3-dimensional colored Doppler image data and the ultrasonic-transmission direction, thereby obtaining Doppler data in which an angle is corrected by a degree of the angle α.

16. The method for acquiring an ultrasonic image according to claim 15, comprising:
alerting the operator that the angle α is equal to or larger than a preset angle in a case where the angle α is equal to or larger than the preset angle.
